(19) United States
(12) Patent Application Publication
(10) Pub. No.: US 2022/0202771 A1
(43) Pub. Date: Jun. 30, 2022

(54) Antidepressants for the Treatment or Prevention of Memory Loss and/or Cognitive Decline or Dysfunction in Aging

(71) Applicant: UNIVERSITY OF NORTH TEXAS HEALTH SCIENCE CENTER AT FORT WORTH, Fort Worth, TX (US)

(72) Inventors: Leigh Johnson, Aledo, TX (US); Sid E. O'Bryant, Aledo, TX (US)

(21) Appl. No.: 17/601,127

(22) PCT Filed: Apr. 2, 2020
(86) PCT No.: PCT/US20/26329

§ 371 Date: Oct. 4, 2021

Related U.S. Application Data
(60) Provisional application No. 62/829,898, filed on Apr. 5, 2019.

(51) Int. Cl. A61K 31/381; A61K 31/137; A61K 31/165; A61K 31/135; A61P 25/28

(52) U.S. Cl. CPC A61K 31/381; A61K 31/137; A61P 25/28; A61K 31/135; A61K 31/165

(57) ABSTRACT

The present invention includes a method of treating or preventing memory loss in a subject suffering from a memory loss-related disease or aging, comprising, consisting essentially of, or consisting of, administering an effective amount of a serotonin-norepinephrine reuptake inhibitor (SNRI) to the subject, wherein the serotonin-norepinephrine reuptake inhibitor is duloxetine or active derivative thereof, such as a subject with memory complaints as part of the aging process, mild cognitive impairment, Alzheimer's disease or dementia and an elevated depressive endophenotype genotype (DepE).

### TECHNICAL FIELD OF THE INVENTION

The present invention relates in general to the field of treating or preventing memory loss and/or cognitive decline or dysfunction, and more particularly, to the use of duloxetine and other antidepressant medications including serotonin-norepinephrine reuptake inhibitor (SNRI) antidepressants for the treatment of memory loss in aging.

### BACKGROUND OF THE INVENTION

Without limiting the scope of the invention, its background is described in connection with memory loss.

Alzheimer's disease (AD) is a major public health crisis. Approximately 12% of adults age 65 and above suffer from AD with an additional approximately 20% suffering from mild cognitive impairment (MCI)—the prodromal phase to AD. Despite the billions of dollars spent, no new drug has been approved for AD or MCI in over 15-years. To date, there has been minimal work to demonstrate the utility of a precision medicine based approach for individualized therapies for older adults suffering from MCI and AD.

What is needed are new approaches to identify agents for use in treating subjects with, or at risk of, memory loss.

### SUMMARY OF THE INVENTION

In one embodiment, the present invention includes a method of treating or preventing memory loss, stabilization of cognition, reduction of cognitive decline in a subject suffering from a memory loss-related to disease or aging or a need to stabilize cognition, and/or reduce cognitive decline, comprising, consisting essentially of, or consisting of, administering an effective amount of a serotonin-norepinephrine reuptake inhibitor to the subject (SNRI). In one aspect, the SNRI is Duloxetine, Atomoxetine, Desvenlafaxine, Levomilnacipran, Milnacipran, Sibutramine, Tramadol, or Venlafaxine, or active derivative thereof. In one aspect, the duloxetine or active derivative thereof is administered to the subject at a dose of 1 to 120 mg/day/person. In another aspect, the duloxetine or active derivative thereof is administered to the subject at a dose of 10-60 mg daily. In another aspect, the duloxetine or active derivative thereof is administered to the subject via oral or parenteral administration. In another aspect, the serotonin-norepinephrine reuptake inhibitor is N-methyl-γ-(1-naphthalenyloxy)-2-thiopropanamine hydrochloride. In another aspect, the duloxetine or active derivative thereof is a racemate, enantiomer, diastereomer, a mixture of enantiomer or a mixture of diastereomer. In another aspect, a pharmaceutically acceptable salt of duloxetine or active derivative thereof is formed from at least one of: an organic acid selected from formic acid, acetic acid, propionic acid, maleic acid, fumaric acid, succinic acid, lactic acid, malic acid, tartaric acid, citric acid, ascorbic acid, malonic acid, oxalic acid, mandelic acid, glycolic acid, phtalic acid, benzenesulphonic acid, toluenesulphonic acid, naphtalenesulphonic acid, or, methanesulphonic acid. In another aspect, the memory loss-related disease is dementia. In another aspect, the dementia is Alzheimer's disease. In another aspect, a reduction in memory loss is in a cognitively normal older adult.

In another embodiment, the present invention includes a method of treating or preventing memory loss, stabilization of cognition, reduction of cognitive decline in a subject suffering from a memory loss-related to disease or aging, or a need to stabilize cognition, and/or reduce cognitive decline comprising, consisting essentially of, or consisting of, administering an effective amount of Duloxetine, Atomoxetine, Desvenlafaxine, Levomilnacipran, Milnacipran, Sibutramine, Tramadol, or Venlafaxine, or active derivative thereof sufficient to reduce the memory loss or cognitive decline. In one aspect, the duloxetine or active derivative thereof is administered to the subject at a dose of 1 to 120 mg/day/person. In another aspect, the duloxetine or active derivative thereof is administered to the subject at a dose of 10-60 mg daily. In another aspect, the duloxetine or active derivative thereof is administered to the subject via oral or parenteral administration. In another aspect, the duloxetine is N-methyl-γ-(1-naphthalenyloxy)-2-thiopropanamine hydrochloride. In another aspect, the duloxetine or active derivative thereof is a racemate, enantiomer, diastereomer, a mixture of enantiomer or a mixture of diastereomer. In another aspect, a pharmaceutically acceptable salt of the duloxetine or active derivative thereof is produced by reacting the duloxetine or active derivative thereof with an inorganic acid, an organic acid, an amino acid, sulfonic acid, an alkali metal or ammonium ion. In another aspect, a pharmaceutically acceptable salt of the duloxetine or active derivative thereof is formed from an organic acid selected from at least one of: formic acid, acetic acid, propionic acid, maleic acid, fumaric acid, succinic acid, lactic acid, malic acid, tartaric acid, citric acid, ascorbic acid, malonic acid, oxalic acid, mandelic acid, glycolic acid, phtalic acid, benzenesulphonic acid, toluenesulphonic acid, naphtalenesulphonic acid, or, methanesulphonic acid. In another aspect, the memory loss-related disease is dementia. In another aspect, the dementia is Alzheimer's disease. In another aspect, a reduction in memory loss is in a cognitively normal older adult. In another aspect, the subject does not have depression, stress-induced incontinence, neuropathic pain or fibromyalgia. In another aspect, the method further comprises the step of identifying a subject with the elevated depressive endophenotype genotype (DepE) and treating the patient with the duloxetine or active derivative thereof to prevent memory loss. In another aspect, the method further comprises the step of identifying a subject with early stage memory loss and providing the subject with the duloxetine or active derivative thereof before true memory impairment exists.

In yet another embodiment, the present invention includes a method for treating or preventing memory loss, stabilization of cognition, reduction of cognitive decline in a patient with a serotonin-norepinephrine reuptake inhibitor, wherein the patient is suffering from mild cognitive impairment (MCI), loss of cognition, and/or reduce cognitive decline, the method comprising, consisting essentially, or consisting, the steps of: determining whether the patient has a DepE endophenotype by asking a specific set of questions regarding depressive symptoms; obtaining or having obtained a biological sample from the patient; and performing or having performed a genotyping assay on the biological sample to determine if the MCI patient also has an elevated depressive endophenotype genotype (DepE); and if the patient has MCI and the DepE endophenotype, then administering the serotonin-norepinephrine reuptake inhibitor to the patient in an amount of 1 to 120 mg/day or less, or if the patient does not have a DepE endophenotype, then not providing the patient with the serotonin-norepinephrine reuptake inhibitor (SNRI). In one aspect, the SNRI is Duloxetine, Atomoxetine, Desvenlafaxine, Levomilnacipran, Milnacipran, Sibutramine, Tramadol, or Venlafaxine, or active derivative thereof. In another aspect, the DepE endophenotype is determined by asking one or more questions selected from memory problems, feeling blue, crying, feeling worthless, and trouble concentrating to determine a DepE score, wherein a positive answer in two or more questions is indicative of a higher DepE score and a lower score is indicative of not having the DepE endophenotype. In another aspect, the DepE endophenotype is determined by use of a software-based computer or hand-held device to administer a patient survey asking one or more questions pertaining to memory problems, feeling blue, crying, feeling worthless, and trouble concentrating to determine a DepE score, wherein a positive answer in two or more questions is indicative of a higher DepE score and a lower score is indicative of not having the DepE endophenotype. In another aspect, the method further comprises patient does not have an ApoEε4 genotype. In another aspect, the method further comprises patient has a DepE scores >=1, >2, >3, >4 or =5. Memory complaint is without a diagnosis of MCI, dementia or Alzheimer's disease. In another aspect, the memory loss is MCI. In another aspect, the memory loss-related disease is dementia. In another aspect, the dementia is Alzheimer's disease. In another aspect, the patient does not have stress-induced incontinence, neuropathic pain or fibromyalgia. In another aspect, the serotonin-norepinephrine reuptake inhibitor is duloxetine or active derivative thereof. In another aspect, the method further comprises the step of varying a dose of the duloxetine or active derivative thereof to the patient, and modifying the dose to that in which memory loss is decreased and side effects are minimized. In another aspect, the method further comprises the step of identifying a subject with the elevated depressive endophenotype genotype (DepE) and treating the patient with the serotonin-norepinephrine reuptake inhibitor to prevent memory loss.

In another embodiment, the present invention includes a non-transitory computer readable medium for detecting or preventing memory loss, stabilization of cognition, reduction of cognitive decline in a patient, comprising instructions stored thereon, that when executed by a computer having a communications interface, one or more databases and one or more processors communicably coupled to the interface and one or more databases, perform the steps comprising: determining whether the patient has a DepE endophenotype by asking a specific set of questions regarding depressive symptoms; obtaining or having obtained a biological sample from the patient or data representative of the biological sample in the database; performing or having performed a genotyping assay on the biological sample to determine if the MCI patient also has an elevated depressive endophenotype genotype (DepE); and at least one of storing or displaying the results obtained thereby. In one aspect, if the patient has MCI and the DepE endophenotype, then administering the serotonin-norepinephrine reuptake inhibitor to the patient in an amount of 1 to 120 mg/day or less, or if the patient does not have a DepE endophenotype, then not providing the patient with the serotonin-norepinephrine reuptake inhibitor. In another aspect, the at least one of storing or displaying the results is on a hand-held device. In another aspect, the DepE endophenotype is determined by asking one or more questions selected from memory problems, feeling blue, crying, feeling worthless, and trouble concentrating to determine a DepE score, wherein a positive answer in two or more questions is indicative of a higher DepE score and a lower score is indicative of not having the DepE endophenotype. In another aspect, the patient does not have an ApoEε4 genotype. In another aspect, the patient has a DepE scores >=1, >2, >3, >4 or =5. In another aspect, the memory complaint is without a diagnosis of MCI, dementia or Alzheimer's disease. In another aspect, the patient has been treated with Duloxetine, Atomoxetine, Desvenlafaxine, Levomilnacipran, Milnacipran, Sibutramine, Tramadol, or Venlafaxine or active derivative thereof.

In another embodiment, the present invention includes a computerized method for detecting or preventing memory loss, stabilization of cognition, reduction of cognitive decline in a patient, comprising: determining whether the patient has a DepE endophenotype by asking a specific set of questions regarding depressive symptoms; obtaining or having obtained a biological sample from the patient or data representative of the biological sample is the database; and performing or having performed a genotyping assay on the biological sample to determine if the MCI patient also has an elevated depressive endophenotype genotype (DepE); and at least one of storing or displaying the results obtained thereby. In one aspect, the at least one of storing or displaying the results is on a hand-held device.

### BRIEF DESCRIPTION OF THE DRAWINGS

For a more complete understanding of the features and advantages of the present invention, reference is now made to the detailed description of the invention along with the accompanying figures and in which:

FIG. 1 is a graph that shows the change in LM scores for LM for Controls—All patients DE scores were high T1 and normal T2. The vertical axis is reflective of scale score points on the Wechsler Memory Scale—Logical Memory Subtest (LMI=immediate memory; LMII=delayed verbal memory). NC=normal control.

FIG. 2 is a graph that shows the results from participants in the treatment group that demonstrates a significant improvement in RBANS Delayed Memory scores over treatment duration whereas there was no significant change in the placebo group (p<0.05).

FIG. 3 is a graph that shows the Ab42 paired sample mean score for a placebo and duloxetine at the time of Ab42 plasma screening and plasma follow-up.

FIG. 4 is a graph that shows the Ab40 paired sample mean score for a placebo and duloxetine at the time of Ab40 plasma screening and plasma follow-up.

FIG. 5 is a graph that shows the Tau paired sample mean score for a placebo and duloxetine at the time of Ab40 plasma screening and plasma follow-up.

FIG. 6 is a graph that shows the NfL paired sample mean score for a placebo and duloxetine at the time of Ab40 plasma screening and plasma follow-up.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

Over the course of many years, the inventors have identified several subgroups of patients suffering from MCI and AD. In this work, the inventors identified a subgroup of patients suffering from MCI and AD, as well as cognitively normal older adults, whose depression seems to be a primary driver of their memory loss. This work has been cross-validated across numerous cohorts. Overall, what the inventors have done is identified a very specific subset of older adults that suffer from a very specific type of depression that is directly related to their memory loss. Most recently, the inventors then conducted a clinical trial to determine if duloxetine (an antidepressant therapy) would result in improved memory among this specific subset of patients. The clinical trial demonstrates a significant benefit of duloxetine on memory abilities among those MCI patients who are elevated in the depressive endophenotype.

Prior work by the present inventors has demonstrated that depression is a risk factor and a prodromal symptom of MCI and AD. Also, depression increased rate for conversion from MCI to AD. However, prior clinical trials using antidepressant medications have yielded mixed results and thus no products have made it to market as therapy to treat memory loss. The solution provided in this work is the identification of the very specific set of older adults who have depression-related memory loss or cognitive decline and are most likely to experience memory enhancement or stabilization of cognition from use of duloxetine and other antidepressant medications, such as serotonin-norepinephrine reuptake inhibitor (SNRI). SNRI include Duloxetine, Atomoxetine, Desvenlafaxine, Levomilnacipran, Milnacipran, Sibutramine, Tramadol, or Venlafaxine, and/or active derivatives thereof. Using this initial study group, the inventors demonstrated the superiority of this precision medicine approach over the “one-size-fits-all” approach to therapeutic interventions that have tried to treat all comers with MCI and AD with antidepressant medications.

The present invention includes a method of treating or preventing memory loss in a subject suffering from a memory loss-related disease or aging, comprising, consisting essentially of, or consisting of, administering an effective amount of a serotonin-norepinephrine reuptake inhibitor to the subject, wherein the serotonin-norepinephrine reuptake inhibitor is duloxetine or active derivative thereof.

As used herein, the phrase “DepE endophenotype” refers to the scoring of a patient's level of geriatric depression or depressive symptoms, wherein a higher DepE score is indicative of having depression (for example a 5 on a 5 point scale), and a score of 0 indicates no DepE symptoms. The DepE evaluation includes one or more questions about cognition selected from: memory problems, feeling blue, crying, feeling worthless, and trouble concentrating, and scoring each on, e.g., a 5-point scale. The inventors have found that the result for DepE have consistently shown that participants who endorse 2 or more of these items score poorer on all cognitive measures, and are twice as likely to have a diagnosis of Mild Cognitive Impairment or Alzheimer's Disease. As such, two or more items that are assessed to determine a high or a low depressive symptom include: memory problems, feeling blue, crying, feeling worthless, and trouble concentrating.

A dosage unit for use of the serotonin-norepinephrine reuptake inhibitor of the present invention, which may be duloxetine or active derivatives thereof, can be provided as a single compound or a mixture with other compounds, excipients, fillers, buffers, etc. The compounds may be mixed together, form ionic or even covalent bonds. The duloxetine or active derivatives thereof, of the present invention may be administered in oral, intravenous (bolus or infusion), intraperitoneal, subcutaneous, intranasal, or intramuscular form, all using dosage forms well known to those of ordinary skill in the pharmaceutical arts. Depending on the particular location or method of delivery, different dosage forms, e.g., tablets, capsules, pills, powders, granules, elixirs, suspensions, syrups, and emulsions may be used to provide the duloxetine or active derivatives thereof, of the present invention to a patient in need of therapy for memory loss.

The duloxetine, or active derivatives thereof, may also be administered as any one of known salt forms. Duloxetine or active derivative thereof is in the form of racemate, enantiomer, diastereomer, a mixture of enantiomer or a mixture of diastereomer. Non-limiting examples of pharmaceutically acceptable salts of duloxetine or active derivative thereof is formed from an organic acid selected from formic acid, acetic acid, propionic acid, maleic acid, fumaric acid, succinic acid, lactic acid, malic acid, tartaric acid, citric acid, ascorbic acid, malonic acid, oxalic acid, mandelic acid, glycolic acid, phtalic acid, benzenesulphonic acid, toluenesulphonic acid, naphtalenesulphonic acid, or, methanesulphonic acid.

The duloxetine, or active derivatives thereof, is typically administered in admixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the duloxetine, or active derivatives thereof, may be formulated to provide, e.g., maximum and/or consistent dosing for the particular form for oral, rectal, topical, intravenous injection, intranasal, or parenteral administration. While the duloxetine, or active derivatives thereof, may be administered alone, it will generally be provided in a stable salt form mixed with a pharmaceutically acceptable carrier. The carrier may be solid or liquid, depending on the type and/or location of administration selected.

Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000, and updates thereto; Martindale, The Extra Pharmacopoeia, Thirty-Second Edition (The Pharmaceutical Press, London, 1999); all of which are incorporated by reference, and the like, relevant portions incorporated herein by reference.

For example, the duloxetine, or active derivatives thereof, may be included in a tablet. Tablets may contain, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with an non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

The duloxetine, or active derivatives thereof, may also be coupled to one or more soluble, biodegradable, bioacceptable polymers as drug carriers or as a prodrug. Such polymers may include: polyvinylpyrrolidone, pyran copolymer, polyhydroxylpropylmethacrylamide-phenol, polyhydroxyethylasparta-midephenol, or polyethyleneoxide-polylysine substituted with palmitoyl residues, mixtures thereof, and the like. Furthermore, the duloxetine, or active derivatives thereof, may be coupled one or more biodegradable polymers to achieve controlled release of the duloxetine, or active derivatives thereof, biodegradable polymers for use with the present invention include: polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels, mixtures thereof, and the like.

In one embodiment, gelatin capsules (gelcaps) may include the duloxetine, or active derivatives thereof, and powdered carriers, such as lactose, starch, cellulose derivatives, magnesium stearate, stearic acid, and the like. Like diluents may be used to make compressed tablets. Both tablets and capsules may be manufactured as immediate-release, mixed-release or sustained-release formulations to provide for a range of release of medication over a period of minutes to hours. Compressed tablets may be sugar coated or film coated to mask any unpleasant taste and protect the tablet from the atmosphere. An enteric coating may be used to provide selective disintegration in, e.g., the gastrointestinal tract.

For oral administration in a liquid dosage form, the oral drug components may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents, mixtures thereof, and the like.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions. Solutions for parenteral administration include generally, a water soluble salt of the active ingredient, suitable stabilizing agents, and if necessary, buffering salts. Antioxidizing agents such as sodium bisulfite, sodium sulfite and/or ascorbic acid, either alone or in combination, are suitable stabilizing agents. Citric acid and its salts and sodium EDTA may also be included to increase stability. In addition, parenteral solutions may include pharmaceutically acceptable preservatives, e.g., benzalkonium chloride, methyl- or propyl-paraben, and/or chlorobutanol. Suitable pharmaceutical carriers are described in Remington's Pharmaceutical Sciences, Mack Publishing Company, a standard reference text in this field, relevant portions incorporated herein by reference.

Capsules. Capsules may be prepared by filling standard two-piece hard gelatin capsules each with 10 to 500 milligrams of powdered active ingredient, 5 to 150 milligrams of lactose, 5 to 50 milligrams of cellulose and 6 milligrams magnesium stearate.

Soft Gelatin Capsules. A mixture of active ingredient is dissolved in a digestible oil such as soybean oil, cottonseed oil or olive oil. The active ingredient is prepared and injected by using a positive displacement pump into gelatin to form soft gelatin capsules containing, e.g., 100-500 milligrams of the active ingredient. The capsules are washed and dried.

Tablets. A large number of tablets are prepared by conventional procedures so that the dosage unit was 100-500 milligrams of active ingredient, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

To provide an effervescent tablet, appropriate amounts of, e.g., monosodium citrate and sodium bicarbonate, are blended together and then roller compacted, in the absence of water, to form flakes that are then crushed to give granulates. The granulates are then combined with the active ingredient, drug and/or salt thereof, conventional beading or filling agents and, optionally, sweeteners, flavors and lubricants.

Injectable solution. A parenteral composition suitable for administration by injection is prepared by stirring 1.5% by weight of active ingredient in deionized water and mixed with, e.g., up to 10% by volume propylene glycol and water. The solution is made isotonic with sodium chloride and sterilized using, e.g., ultrafiltration.

Suspension. An aqueous suspension is prepared for oral administration so that each 5 ml contain 100 mg of finely divided active ingredient, 200 mg of sodium carboxymethyl cellulose, 5 mg of sodium benzoate, 1.0 g of sorbitol solution, U.S.P., and 0.025 ml of vanillin.

For mini-tablets, the active ingredient is compressed into a hardness in the range 6 to 12 Kp. The hardness of the final tablets is influenced by the linear roller compaction strength used in preparing the granulates, which are influenced by the particle size of, e.g., the monosodium hydrogen carbonate and sodium hydrogen carbonate. For smaller particle sizes, a linear roller compaction strength of about 15 to 20 KN/cm may be used.

Kits. The present invention also includes pharmaceutical kits useful, for example, for the treatment or prevention of memory loss, which comprise one or more containers containing a pharmaceutical composition comprising a therapeutically effective amount of duloxetine, or active derivatives thereof. Such kits may further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, may also be included in the kit. It should be understood that although the specified materials and conditions are important in practicing the invention, unspecified materials and conditions are not excluded so long as they do not prevent the benefits of the invention from being realized.

Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

As used herein, the term “chewable” refers to semi-soft, palatable and stable chewable treat without addition of water. It should be appreciated to the skilled artisan that a chewable composition will be stable and palatable, fast disintegrating, semi-soft medicated chewable tablets (treats) by extrusion without the addition of extraneous water. A soft chewable tablet does not harden on storage and are resistant to microbial contamination. A semi-soft chewable contain a blend of any one or more of binders, flavors, palatability enhancers, humectants, disintegrating agents, non-aqueous solvents, and diluents that are plasticized with liquid plasticizers, such as glycols and polyols to make them ductile and extrudable. The chewable can be made by extrusion, e.g., including fats or lipids as plasticizers and binding agents, is manufactured in the absence of added water, uses plasticizers to replace water in extrudable matrices, contains humectants to maintain the extrudable chew in a pliant and soft state during its shelf life, or any combination thereof. The chewable form may be provided in conjunction with one or more flavorants and/or taste masking agents that improve the taste of the formulation greater than 10, 20, 30, 40, 50, 60, 70, 80, or 90%. The chewable can include the active agent and the ion exchange resin to enhance taste masking.

A Depressive Endophenotype of Memory Loss—a specific type of depression that is linked to cognitive loss, mild cognitive impairment (MCI) and Alzheimer's disease (AD).

In an initial study from over 500 participants from a multi-ethnic rural cognitive aging study, the inventors identified a specific set of depressive symptoms from the Comprehensive Geriatric Assessment (GDS-30) that was significantly associated with MCI risk. This depressive endophentoype of cognitive dysfuncion (DepE) was generated as follows. The study population was randomly split into two groups, training and test set (each group containing both participants with normal cognition and those with mild cognitive impairment). Next, in the training set, descriptive analyse were conducted to determine the specific depressive symptoms that were overexpressed among MCI cases as compared to controls. Those specific depressive symptoms were then combined into a single scale, the Depressive Endophenotype (DepE). Next, the DepE scale (i.e. the specific symptoms of depression related to memory loss) was then applied to the test sample to determine if DepE would predict MCI risk. DepE significantly increased risk for MCI diagnosis (odds ratio [OR]=2.04; 95% Confidence Interval (CI)=1.54-2.69), which was the only significant predictor aside from age (OR=1.09; 95% CI=1.05-1.13) and education (OR=0.82; 95% CI=0.71-0.95). Of note, GDS total scores (minus DepE items) were not significantly related to MCI status with DepE scores entered into the model. Therefore, DepE scores and not global depression scores were specifically related to MCI risk. ApoEε4 genotype (the strongest genetic risk for MCI and AD) did not enter the model²⁹. Analyses also demonstrate that DepE is not simply a surrogate of subjective cognitive dysfunction, which alone does not significantly predict MCI risk among this population. Therefore, DepE identifies the very specific subset of depressive symptoms associated with memory/cognitive loss thereby isolating a specific subset of individuals whose memory loss or cognitive dysfunction is likely due to a specific type of depression.

Next, the DepE was applied to the Texas Alzheimer's Research and Care Consortium (TARCC) cohort. A logistic regression model was created with AD versus normal control as the outcome variable; age, gender, education, ApoEε4 presence (yes/no), GDS total score and DepE scores entered as the predictor variables. Age (OR=1.18, 95% CI=1.12-1.24, p<0.001), ApoEε4 status (OR=2.42, 95% CI=1.13-5.19, p=0.02) and the DepE scores (OR=2.49, 95% CI=1.40-4.43, p=0.002) were the only significant predictors of AD status. Importantly, clinical covariates (diabetes, hypertension, obesity) did not contribute to the model. In the forward conditional stepwise logistic regression, the order of entry into the model was age, DepE scores, and finally ApoEε4 status. DepE score alone was a significant predictor of AD status using receiver operating characteristic (ROC) curve analysis (Area Under the Curve [AUC]=0.74 (95% CI=0.68-0.81), p<0.001)²⁹.

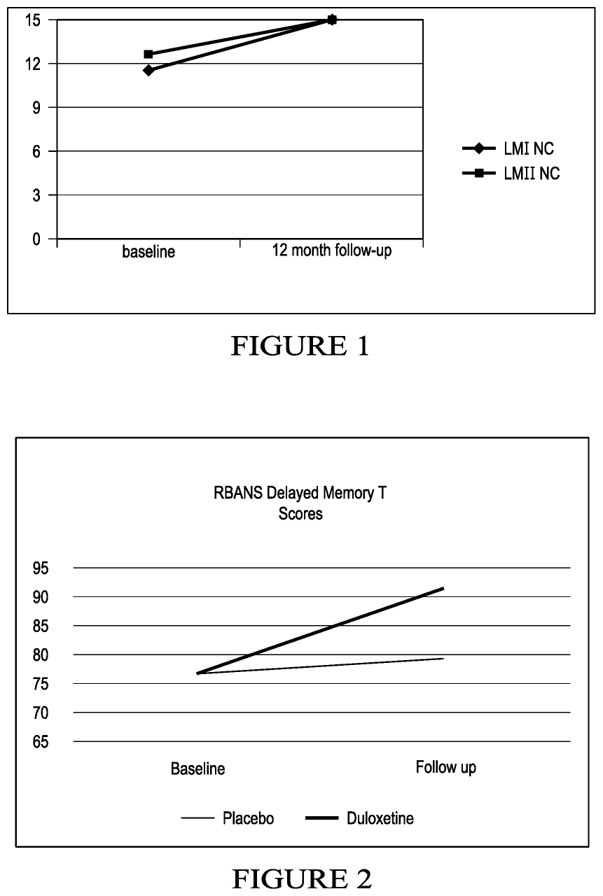

Longitudinal Analyses. Baseline DepE scores were significantly related to decline in memory and global cognition as well as increased disease progression longitudinally²⁹. FIG. 1 demonstrates that improvement in DepE scores over time also was associated with a significant improvement in verbal memory scores (Logical Memory I and II). Next, the inventors analyzed data from the Western Australia Memory Study cohort and found that, among those ages 65 and above, elevations in DepE scores were significantly related to poorer cognitive functioning (OR=1.53; 95% CI=1.01-2.32, p=0.04). Among those 70 and above, elevations in DepE scores were the single strongest risk for poorer cognitive functioning (OR=2.23, 95% CI 1.12-4.40, p=0.02) with neither age nor education being significant³¹. Therefore, DepE is associated with poorer memory among cognitively normal older adults as well as those diagnosed with MCI and AD.

Depression as a risk factor for cognitive loss among Mexican Americans³²⁻³⁴. The inventors specifically studied depression and cognition among Mexican Americans. In their prior work, the inventors found that, when compared to non-Hispanic whites: depression levels were higher among Mexican Americans³², depression symptoms were consistently associated with MCI risk among Mexican Americans and depression is more strongly associated with poorer cognition among Mexican Americans^{30,33-36}.

Next, the inventors examined data from n=317 Mexican American participants from the inventors HABLE study. Those individuals with DepE>=2 had lower scores on memory and global cognition and were more likely to decline over 12-months. Those participants with DepE>=2 were significantly more likely to have a diagnosis of MCI. Therefore, as with non-Hispanic whites, higher DepE scores among Mexican Americans were associated with poorer memory scores and increased likelihood of MCI diagnosis in addition to increased risk for memory decline³⁰.

Use of the depressive endophenotype for a precision medicine approach to improving memory among specific MCI cases.

Next, the inventors conducted an investigator-initiated clinical trial (NCT02590874). This was a randomized, double-blind, placebo-controlled study of the feasibility and safety of using an antidepressant therapy, specifically a serotonin-norepinephrine reuptake inhibitor, to improve memory and stabilize cognition in individuals with a diagnosis of MCI confirmed by a comprehensive neuropsychological battery. Duloxetine, a serotonin-norepinephrine reuptake inhibitor that is generally well tolerated and beneficial in older adults suffering from major depressive disorder, was selected as the study drug. Based on study data, the inventors determined that duloxetine may be used for treating or preventing memory loss and cognitive decline among a specific subset of MCI cases with elevated DepE scores. Treatment duration was 4 months of duloxetine versus placebo. The initial dosage consisted of 30 mg/day duloxetine or placebo; after two weeks, dosage was increased to 60 mg/day. Dosage during the last two weeks of the 4-month study was reduced to 30 mg/day. Secondary endpoints of change in Repeatable Battery for the Assessment of Neuropsychological Status (RBANS) memory scores from baseline to study completion were analyzed. In this study, the inventors recruited n=16 participants with clinical diagnosis of amnestic MCI (single or multi-domain). N=14 subjects enrolled were of Mexican American ethnicity. The findings were: (1) it is possible to effectively enroll MCI cases with DepE scores >=2, (2) no Serious Adverse Effects (SAEs) were identified and therefore supporting safety. With regards to change in RBANS scores, at baseline there was no significant difference in RBANS Delayed Memory scores between duloxetine and placebo groups (see Table 1). However, participants in the treatment group demonstrated a significant improvement in RBANS Delayed Memory scores over treatment duration whereas there was no significant change in the placebo group (p<0.05) (FIG. 2).

TABLE 1
RBANS scores at baseline and follow-up.

| | Baseline | Follow-up |
|---|---|---|
| RBANS Total | | |
| Placebo | 69.00(10.87) | 70.37 (10.82) |
| Duloxetine | 72.37(5.12) | 80.37(5.31)* |
| RBANS Immediate Memory | | |
| Placebo | 70.00(8.76) | 73.75(10.88) |
| Duloxetine | 74.62(11.17) | 88.75(11.43)* |
| RBANS Delayed Memory | | |
| Placebo | 76.75(19.01) | 79.25(14.63) |
| Duloxetine | 76.63(11.01) | 91.50(6.98)* |

*p < .05

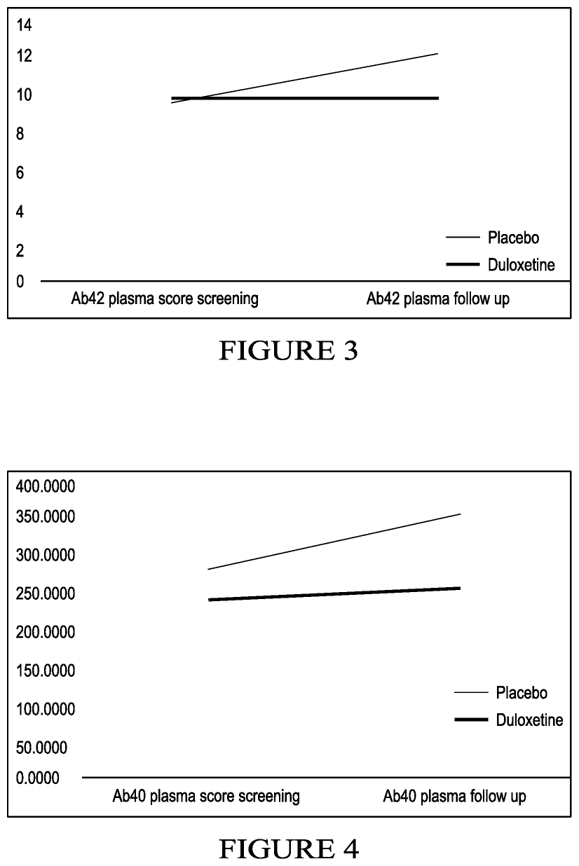

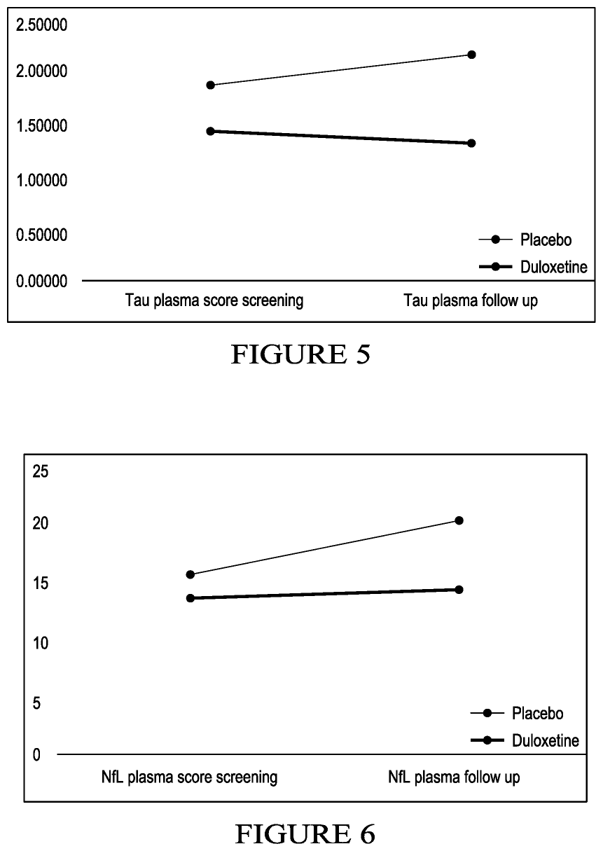

Fasting blood samples were collected at baseline and follow-up. Several plasma biomarkers were assessed to determine neurodegeneration over treatment duration: Neurofilament light (NfL)(FIG. 6), Tau (FIG. 5), Aβ40 (FIG. 4) and Aβ42 (FIG. 3). At baseline, no significant differences were found among the treatment and placebo groups. At follow-up among the placebo group, significant increases compared to baseline were observed among Aβ40 (p<0.05) and Aβ42 (p<0.05). Increases in markers of Tau and NfL were approaching significant. At follow-up among the treatment group, markers of neurodegeneration were primarily stable and no significant differences were observed between baseline and follow-up measures for the amyloid biomarkers Aβ40 and Aβ42 (p<0.05), showing that the antidepressant therapy stabilized neurodegeneration during treatment duration in the subset of MCI cases with elevated DepE scores.

Thus, serotonin-norepinephrine reuptake inhibitors such as duloxetine (or a derivative of this medication) can be marketed and sold for improved memory and stabilization of neurodegeneration (including preventing cognitive decline or stabilizing cognitive decline), including among a very specific subset of older adults with depression-related memory loss or cognitive dysfunction. The medication can be used to treat MCI and AD as well as prevent decline among cognitively normal older adults. Further, using the DepE endophenotypes, a software product (such as a hand-held device application) can be used in primary care setting by physicians to quickly determine if any particular patient fits the depressive endophenotype and then the physician can prescribe the medication.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

Johnson L A, Hall J R & O'Bryant S E (2013). A Depressive Endophenotype of Mild Cognitive Impairment and Alzheimer's Disease. PLoS ONE, 8(7). doi:10.1371/journal.pone.0068848.
Johnson L A, Gamboa A, Vintimilla R, Edwards M, Hall J, Weiser B, Yadav M, Dickensheets T & O'Bryant S E (2016). A Depressive Endophenotype for Predicting Cognitive Decline among Mexican American Adults and Elders. Journal of Alzheimer's Disease, 54(1), 201-206.PMID: 27472872.
Johnson L A, Sohrabi H R, Hall J R, Taddei K, Edwards M, O'Bryant S E & Martins R N (2015). A Depressive Endophenotype of Poorer Cognition among Cognitively Healthy Community-Dwelling Adults: Results from the Western Australia Memory Study. International Journal of Geriatric Psychiatry, 30(8), 881-6. PMID: 25394326 DOI: 10.1002/gps.4231.

1. A method of treating or preventing memory loss, stabilization of cognition, reduction of cognitive decline in a subject suffering from a memory loss-related disease or aging, or a need to stabilize cognition, and/or reduce cognitive decline comprising administering an effective amount of a serotonin-norepinephrine reuptake inhibitor to the subject, wherein the amount of the serotonin-norepinephrine reuptake inhibitor (SNRI) is effective to treat or prevent memory loss, stabilize cognition, reduce cognitive decline.

2. The method of claim 1, wherein the SNRI is Duloxetine, Atomoxetine, Desvenlafaxine, Levomilnacipran, Milnacipran, Sibutramine, Tramadol, or Venlafaxine, or active derivative thereof.

3. The method of claim 1, wherein the SNRI is duloxetine or active derivative thereof and is at least one of:
administered to the subject at a dose of 1 to 120 mg/day/person;
administered to the subject at a dose of 10-60 mg daily; or
administered to the subject via oral or parenteral administration.

4. (canceled)

5. (canceled)

6. The method of claim 1, wherein the serotonin-norepinephrine reuptake inhibitor is N-methyl-γ-(1-naphthalenyloxy)-2-thiopropanamine hydrochloride, or a racemate, enantiomer, diastereomer, a mixture of enantiomer or a mixture of diastereomers.

7. (canceled)

8. The method of claim 2, wherein a pharmaceutically acceptable salt of the SNRI or active derivative thereof is formed from at least one of: an organic acid selected from formic acid, acetic acid, propionic acid, maleic acid, fumaric acid, succinic acid, lactic acid, malic acid, tartaric acid, citric acid, ascorbic acid, malonic acid, oxalic acid, mandelic acid, glycolic acid, phtalic acid, benzenesulphonic acid, toluenesulphonic acid, naphtalenesulphonic acid, or, methanesulphonic acid.

9. The method of claim 1, wherein the SNRI is provided in an amount sufficient to stabilize neurodegeneration or cognitive decline in DepE subjects having at least one of cognitive decline, mild-cognitive impairment, or Alzheimer's disease.

10. The method of claim 1, wherein the memory loss-related disease is dementia, or Alzheimer's disease; or the reduction in memory loss is in a cognitively normal older adult.

11. (canceled)

12. A method of treating or preventing memory loss, stabilization of cognition, reduction of cognitive decline in a subject suffering from a memory loss-related disease or aging, or a need to stabilize cognition, and/or reduce cognitive decline comprising, administering an effective amount of Duloxetine, Atomoxetine, Desvenlafaxine, Levomilnacipran, Milnacipran, Sibutramine, Tramadol, or Venlafaxine or active derivative thereof sufficient to reduce the memory loss.

13. The method of claim 12, wherein the duloxetine or active derivative thereof is at least one of:
administered to the subject at a dose of 1 to 120 mg/day/person;
administered to the subject at a dose of 10-60 mg daily; or
administered to the subject via oral or parenteral administration.

14. (canceled)

15. (canceled)

16. The method of claim 12, wherein the duloxetine is N-methyl-γ-(1-naphthalenyloxy)-2-thiopropanamine hydrochloride, or a racemate, enantiomer, diastereomer, a mixture of enantiomer or a mixture of diastereomers.

17. (canceled)

18. The method of claim 12, wherein a pharmaceutically acceptable salt of the duloxetine or active derivative thereof is produced by reacting the duloxetine or active derivative thereof with an inorganic acid, an organic acid, an amino acid, sulfonic acid, an alkali metal or ammonium ion.

19. The method of claim 12, wherein a pharmaceutically acceptable salt of the duloxetine or active derivative thereof is formed from an organic acid selected from at least one of: formic acid, acetic acid, propionic acid, maleic acid, fumaric acid, succinic acid, lactic acid, malic acid, tartaric acid, citric acid, ascorbic acid, malonic acid, oxalic acid, mandelic acid, glycolic acid, phtalic acid, benzenesulphonic acid, toluenesulphonic acid, naphtalenesulphonic acid, or, methanesulphonic acid.

20. The method of claim 12, wherein the Duloxetine, Atomoxetine, Desvenlafaxine, Levomilnacipran, Milnacipran, Sibutramine, Tramadol, or Venlafaxine or active derivative thereof, is provided in an amount sufficient to stabilize neurodegeneration or cognitive decline in DepE subjects having at least one of cognitive decline, mild-cognitive impairment, or Alzheimer's disease.

21. The method of claim 12, wherein the memory loss-related disease is dementia or Alzheimer's disease; or a reduction in memory loss is in a cognitively normal older adult.

22. (canceled)

23. The method of claim 12, wherein the subject does not have depression, stress-induced incontinence, neuropathic pain or fibromyalgia.

24. The method of claim 12, further comprising the step of identifying a subject with the elevated depressive endophenotype genotype (DepE) and treating the patient with the duloxetine or active derivative thereof to prevent memory loss; or identifying a subject with early stage memory loss and providing the subject with the duloxetine or active derivative thereof before true memory impairment exists.

25. (canceled)

26. A method for treating or preventing memory loss, stabilization of cognition, reduction of cognitive decline in a patient with a serotonin-norepinephrine reuptake inhibitor, wherein the patient is suffering from mild cognitive impairment (MCI), loss of cognition, and/or reduce cognitive decline, the method comprising the steps of:
determining whether the patient has a DepE endophenotype by asking a specific set of questions regarding depressive symptoms;
obtaining or having obtained a biological sample from the patient; and
performing or having performed a genotyping assay on the biological sample to determine if the MCI patient also has an elevated depressive endophenotype genotype (DepE); and
if the patient has MCI and the DepE endophenotype, then administering the serotonin-norepinephrine reuptake inhibitor (SNRI) to the patient in an amount sufficient to at least one of: treat or prevent memory loss, stabilize cognition, or reduce cognitive decline.

27. The method of claim 26, wherein the DepE endophenotype is determined by asking one or more questions selected from memory problems, feeling blue, crying, feeling worthless, and trouble concentrating to determine a DepE score, wherein a positive answer in two or more questions is indicative of a higher DepE score and a lower score is indicative of not having the DepE endophenotype.

28. The method of claim 26, wherein the patient at least one of: does not have an ApoEε4 genotype; has a DepE scores >=1, >2, >3, >4 or =5; or does not have stress-induced incontinence, neuropathic pain or fibromyalgia.

29. (canceled)

30. The method of claim 26, wherein the memory complaint is without a diagnosis of MCI, dementia or Alzheimer's disease.

31. The method of claim 26, wherein the memory loss is MCI, dementia, or Alzheimer's disease.

32. (canceled)

33. The method of claim 26, wherein the SNRI is Duloxetine, Atomoxetine, Desvenlafaxine, Levomilnacipran, Milnacipran, Sibutramine, Tramadol, or Venlafaxine or active derivative thereof or active derivative thereof.

34. The method of claim 33, further comprising the step of varying a dose of the duloxetine or active derivative thereof to the patient, and modifying the dose to that in which memory loss is decreased and side effects are minimized; or identifying a subject with the elevated depressive endophenotype genotype (DepE) and treating the patient with the serotonin-norepinephrine reuptake inhibitor to prevent memory loss.

35. (canceled)

36. A non-transitory computer readable medium for detecting, preventing or treating memory loss, stabilization of cognition, reduction of cognitive decline in a patient, comprising instructions stored thereon, that when executed by a computer having a communications interface, one or more databases and one or more processors communicably coupled to the interface and one or more databases, perform the steps comprising:
determining whether the patient has a DepE endophenotype by asking a specific set of questions regarding depressive symptoms;
obtaining or having obtained a biological sample from the patient or data representative of the biological sample is the database;
performing or having performed a genotyping assay on the biological sample to determine if the MCI patient also has an elevated depressive endophenotype genotype (DepE); and
at least one of storing or displaying the results obtained thereby for the DepE endophenotype, and optionally the computerized method is performed on a hand-held device.

37. (canceled)

38. The non-transitory computer readable medium of claim 36, wherein if the patient has MCI and the DepE endophenotype, then administering the serotonin-norepinephrine reuptake inhibitor to the patient in an amount of 1 to 120 mg/day or less, or if the patient does not have a DepE endophenotype, then not providing the patient with the serotonin-norepinephrine reuptake inhibitor.

39. The non-transitory computer readable medium of claim 36, wherein the DepE endophenotype is determined by asking one or more questions selected from memory problems, feeling blue, crying, feeling worthless, and trouble concentrating to determine a DepE score, wherein a positive answer in two or more questions is indicative of a higher DepE score and a lower score is indicative of not having the DepE endophenotype.

40. The non-transitory computer readable medium of claim 36, wherein the memory complaint is without a diagnosis of MCI, dementia or Alzheimer's disease.

41. The non-transitory computer readable medium of claim 36, wherein if the patient if a DepE endophenotype is obtained, treating the patient with Duloxetine, Atomoxetine, Desvenlafaxine, Levomilnacipran, Milnacipran, Sibutramine, Tramadol, or Venlafaxine or active derivative thereof.

42. A computerized method for detecting for detecting, preventing or treating memory loss, stabilization of cognition, reduction of cognitive decline in a patient, comprising:
determining whether the patient has a DepE endophenotype by asking a specific set of questions regarding depressive symptoms;
obtaining or having obtained a biological sample from the patient or data representative of the biological sample is the database;
performing or having performed a genotyping assay on the biological sample to determine if the MCI patient also has an elevated depressive endophenotype genotype (DepE); and
at least one of storing or displaying the results obtained thereby for the DepE endophenotype, and optionally the computerized method is performed on a hand-held device.

43. (canceled)
